(19) United States
(12) Patent Application Publication
(10) Pub. No.: US 2013/0171702 A1
(43) Pub. Date: Jul. 4, 2013

(54) METHOD FOR CULTURING MIXOTROPHIC UNICELLULAR ALGAE IN THE PRESENCE OF A DISCONTINUOUS SUPPLY OF LIGHT IN THE FORM OF FLASHES

(71) Applicant: Pierre Calleja, Bordeaux (FR)

(72) Inventor: Pierre Calleja, Bordeaux (FR)

(73) Assignee: FERMENTALG, Libourne (FR)

(21) Appl. No.: 13/822,805

(22) PCT Filed: Sep. 15, 2011
(86) PCT No.: PCT/FR11/52114

§ 371 Date: Mar. 13, 2013

(30) Foreign Application Priority Data
Sep. 15, 2010 (FR) 1057380

(51) Int. Cl. C12P 7/64

(52) U.S. Cl. CPC C12P 7/64
USPC 435/134; 435/257.1; 435/252

(57) ABSTRACT

A novel method for culturing mixotrophic single-cell algae makes it possible to enrich the lipid content of these algae, the enrichment being induced by a variable or discontinuous provision of light, in particular in the form of flashes.

The present invention relates to a novel method for culturing unicellular algae (microalgae) under mixotrophic conditions, in particular permitting enrichment of the lipid content of these algae.

This method is based on supply of light to the culture medium that is variable or discontinuous over time, said supply inducing an increase in the yield of the cultures and the lipid content of the algae.

### Preamble

The unicellular algae are currently the subject of numerous industrial projects with a view to their direct use, as a food supplement, or indirect use as a raw material for green chemistry.

The lipids obtained from these microalgae are particularly sought after as they have excellent nutritional qualities. In particular, they contain essential long-chain polyunsaturated fatty acids (PUFA), such as EPA and DHA, which are used, in particular, in the formulation of substitutes for human milk. In aquaculture, the microalgae serve as substitutes for fish oils and fish meal.

On the basis that under favourable conditions the microalgae can accumulate up to 80% of their dry weight in fatty acids, the latter offer a credible alternative to the growing of oleaginous terrestrial plants for the production of biofuels [Li, Y. et al., 2008 Biotechnol. Prog., 24: 815-820].

The unicellular algae are photosynthetic microorganisms of autotrophic character, i.e. they have the capacity to grow autonomously by photosynthesis.

Most species of unicellular algae encountered in fresh water or in the oceans are strictly autotrophic, i.e. they cannot grow other than by photosynthesis. For the latter, the presence of carbon-containing substrates or of organic matter in their environment is not favourable to them, and even tends to inhibit their growth.

However, a certain number of species of unicellular algae, of very varied families and origin, are found to be not strictly autotrophic. Some of them, called heterotrophs, are capable of developing in the complete absence of light, by fermentation, i.e. by utilizing organic matter.

Other species of algae, for which photosynthesis remains indispensable for their development, are capable of using both photosynthesis and organic matter present in their environment. These intermediate species, called mixotrophs, can be cultured both in the presence of light and of organic matter.

This particular feature of the so-called “mixotrophic” algae seems to be connected with their metabolism, which allows them to carry on photosynthesis and fermentation simultaneously. The two types of metabolism coexist with an overall positive effect on the growth of the algae [Yang C. et al. (2000) Biochemical Engineering Journal 6: 87-102].

At present, the classification of algae is still based largely on morphological criteria and on the nature of the photosynthetic pigments that they contain. This classification gives little information about the autotrophic, heterotrophic or mixotrophic character, whereas algae cover a very great diversity of species and forms [Dubinsky et al. 2010, Hydrobiologia, 639: 153-171].

Therefore, a strain is considered to be mixotrophic if it can be proved experimentally that it has the ability to grow by photosynthesis in a mineral medium, to which a carbon-containing substrate is added, such as glucose, acetate or glycerol. If this supplementation with carbon-containing substrate does not give rise to inhibition of growth during the illuminated phase, the strain can be considered to be mixotrophic.

Although, for certain species of algae, being able to grow under mixotrophic conditions makes it possible theoretically to achieve a better yield in culture with respect to cellular biomass and production of lipids [Ceron Garcia M. C. et al. 2000, Journal of Applied Phycology, 13: 239-248], such a level of yield is only rarely attained.

In fact, management of the ratio of the amount of light to the amount of carbon-containing substrate to be added to the culture medium in order to obtain an optimum yield is still difficult to determine [Lee Y. K. et al. 2001, Journal of Applied Phycology, 13: 307-315], in particular regarding the production of a commercially useful amount of lipids.

Therefore, there is still a need to improve the existing culture techniques for mixotrophic microalgae in order to obtain production conditions providing a high level of biomass and high concentrations of lipids.

Studies carried out on different species of microalgae have established that light intensity has a direct influence on the quantitative and qualitative yield of cultures carried out in mixotrophic mode.

Thus, light intensity equal to that used in autotrophic mode is generally too strong for growing algae in mixotrophic mode. Because of this phenomenon of photo-inhibition, it is often advisable to reduce the light intensity in order to obtain better yields [Liang Y. et al., 2009, Biotechnol. Lett., 31: 1043-1049; Chojnacka, K. et al., 2004, Enzyme and Microbial Technology, 34: 461-465; Bouarab L. et al., 2004, Water Research, 38: 2706-2712; Jeon Y. C. et al. 2006, Enzyme and Microbial Technology, 39: 490-495].

Because on an industrial scale algae are often cultured in the open, i.e. with natural illumination, experiments carried out in order to determine the optimum conditions for culture in mixotrophic mode are generally carried out with continuous phases of illumination. In certain cases an illuminated phase alternates with a darkness phase daily (photoperiod), thus reproducing day and night. However, there is no mention in the prior art of repeated alternations or of significant variations over time of the light intensity supplied to the cultures.

Previous experiments with cultures with alternating culture phases in autotrophic mode (illuminated) and heterotrophic mode (in darkness), each of several hours, proved disappointing in terms of optimization of the cultures, because the algae needed to adapt to the changes in culture conditions. A lag time was observed at the start of each cycle, connected with the so-called photo-activation phenomenon, contributing to a drop in yield of the cultures [Ogbonna J. C. et al., 1997, Journal of Applied Phycology 9: 359-366].

This is undoubtedly the reason why the authors of the various works for optimization of the culture of algae in mixotrophic mode did not consider that an alternation of illuminated and dark phases could have a favourable effect on the yield of the cultures.

Surprisingly, the inventor found, on the contrary, that a discontinuous supply of light was not detrimental to the yield of cultures carried out in mixotrophic mode. In particular, he observed that by varying the light intensity, either by a rapid alternation of dark phases and illuminated phases (flashes), or by making the light intensity fluctuate over time, he could act positively on the yield of the cultures in terms of biomass, and more particularly on the lipid content of the cells.

Based on these observations, the inventor has developed a method for culturing mixotrophic algae that makes it possible to achieve, on the one hand, an increase in biomass, and on the other hand to enrich the lipid content of the algae. This method is based on a light supply that is variable or discontinuous over time.

It appears that this method, the subject of the present application, can be generalized for numerous species of mixotrophic unicellular algae.

The various aspects and advantages of the invention are detailed below.

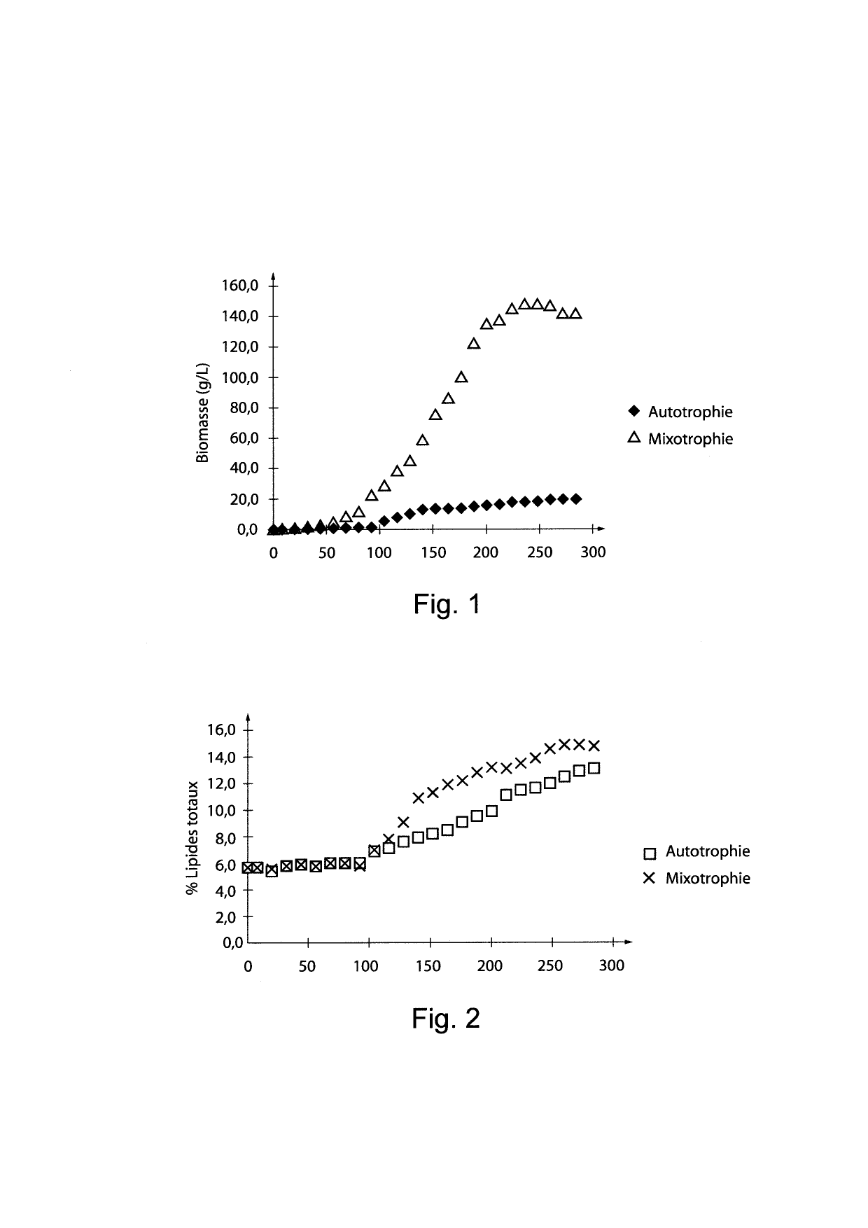

FIG. 1: Graph comparing the biomass of cultures of Tetraselmis carried out respectively in mixotrophic mode with light supplied in the form of flashes according to the invention (Δ) and in autrotrophic mode (♦) i.e. in continuous light.

FIG. 2: Graph comparing the lipid content of cells of Tetraselmis cultured respectively in mixotrophic mode with light supplied in the form of flashes according to the invention (X) and in autrotrophic mode (□).

### DETAILED DESCRIPTION

The present invention thus relates to a method for culturing unicellular algae making it possible to increase their biomass and to enrich their lipid content.

This method also makes it possible to select strains of microalgae that are particularly adapted to the production of lipids in mixotrophic mode.

This method is characterized in that the luminous flux supplied to the algae in culture is variable or discontinuous over time.

In contrast to conventional wisdom, it was found that variable or discontinuous illumination of the cultures, in particular in mixotrophic mode, had a favourable effect on the development of the algae and made it possible to increase the production of lipids by the latter.

Without being bound by theory, the inventor thinks that discontinuous or variable supply of light has the effect of causing stress in the algae that is favourable to the synthesis of lipids. In fact, it frequently happens in nature that algae accumulate lipid reserves so as to be able to withstand environmental stresses.

By “discontinuous illumination” is meant illumination punctuated by periods of darkness. The periods of darkness can occupy more than a quarter of the time, preferably half of the time or more, during which the algae are cultured.

According to a preferred aspect of the invention, the discontinuous illumination is supplied in the form of flashes, i.e. for periods of short duration. The successive phases of illumination are then generally between 5 seconds and 10 minutes, preferably between 10 seconds and 2 minutes, more preferably between 20 seconds and 1 minute.

By “variable illumination” is meant a supply of light the intensity of which is varied deliberately over time, cyclically or non-cyclically.

According to the invention, the illumination can vary continuously, i.e. the light intensity is not constant but varies continually over time (dμmol(photons)/dt≠0).

According to the invention, it is also possible to use a light supply combining continuous and discontinuous phases of illumination.

The invention relates, in particular, to a method for culturing unicellular algae, characterized in that said algae are cultured in darkness with a supply of light that is discontinuous or variable over time, the intensity of which, in micromoles of photons, varies with an amplitude greater than or equal to 10 μmol. m⁻². s⁻¹ at a rate of several times per hour.

What these various modes of illumination, discontinuous or variable, have in common is that, according to the invention, the light intensity supplied to the algae in culture, expressed in micromoles of photons per second per square metre (μmol. m⁻². s⁻¹), varies several times in just one hour, with an amplitude generally greater than 8 μmol. m⁻². s⁻¹, preferably greater than or equal to 10 μmol. m⁻². s⁻¹, more preferably greater than or equal to 15 μmol. m⁻². s⁻¹. In other words, the light intensity reaches, each hour, preferably several times an hour, a high value and a low value, the difference of which is greater than or equal to that stated above. Preferably, said light intensity reaches each hour, successively the values (i.e. passes through these values): 2 μmol. m⁻². s⁻¹ and 10 μmol. m⁻². s⁻¹, more preferably the values 0 μmol. m⁻². s⁻¹ and 50 μmol. m⁻². s⁻¹, even more preferably the values 0 and 20 μmol. m⁻². s⁻¹.

It should be noted that 1 μmol. m⁻². s⁻¹ corresponds to 1 μE m⁻². s⁻¹ (Einstein), the unit used in the examples in the present application.

According to a preferred aspect of the invention, the light intensity varies between the values 0 and 20 μmol. m⁻². s⁻¹, preferably between 0 and 50 μmol. m⁻². s⁻¹.

The supply of light to the cultures can be provided by lamps distributed around the external wall of the fermenters. A clock switches on these lamps for defined illumination times. The fermenters are preferably located in a chamber shielded from daylight, the ambient temperature of which can be controlled.

The method according to the invention applies more particularly to unicellular algae capable of growing under mixotrophic conditions.

As pointed out in the preamble of the present application, a culture of algae in mixotrophic mode is defined as a culture carried out in autotrophic mode in a culture medium enriched with carbon-containing substrates.

Preferably, said carbon-containing substrates comprise, or consist of, acetate, glucose, cellulose, starch, lactose, saccharose or glycerol.

Within the meaning of the present invention, a species of alga is regarded as mixotrophic provided it can be cultured in the light, in a minimum medium (for example MM or f/2) to which a carbon-containing substrate is added at the rate, for example, of a concentration of carbon, glycerol or acetate, equivalent to or greater than 5 mM, without observing inhibition of growth, i.e. without finding a loss of biomass in dry matter relative to a culture carried out in the identical minimum medium lacking carbon-containing substrate (i.e. in autotrophic mode).

For the purposes of the present invention, mixotrophic microalgae are preferably used, in which at least 25%, preferably at least 50% of the energy they produce is derived from the utilization of said carbon-containing substrate.

The culture medium must contain a quantity of carbon-containing substrate sufficient for fermentation, but not too high, in order to avoid inhibiting growth of the algae. Preferably, the culture medium according to the invention comprises an available glucose concentration below 10 g/L, preferably between 4 and 6 g/L.

According to a preferred aspect of the invention the carbon-containing substrate comprises acetate, preferably sodium acetate, the concentration of which in the culture medium is generally comprised between 5 mM and 50 mM, preferably between 15 and 25 mM.

Advantageously, the species of mixotrophic algae is selected from the following classes: Euglenophyceae, Prasinophyceae, Eustigmatophyceae, Bacillariophyceae, Prymnesiophyceae, Prymnesiophyceae, Pinguiophyceae, Eustigmatophyceae, Bacillariophyceae, Dinophyceae, Trebouxiophyceae, Bicosoecophyceae, Katablephariophyceae, Dinophyceae, Chlorophyceae, Haptophyceae, Raphidophyceae, Chysophyceae, Coscinodiscophyceae, Alveolata, Bangiophyceae, Rhodophyceae, in particular lipid-producing strains, and preferably strains producing at least 5% of lipids in autotrophic mode.

The microalgae belonging to the Prasinophyceae class are preferably of the genus Tetraselmis sp.

A culture method according to the invention is carried out for example under mixotrophic conditions in the presence of light supplied in the form of flashes of light, preferably between 20 and 30 flashes per hour, the intensity of which is generally comprised between 5 and 50 μmol. m⁻². s⁻¹, preferably between 5 and 15 μmol. m⁻². s⁻¹.

According to a preferred example of the invention, 30 flashes of 30 seconds per hour with an intensity of about 10 μmol. m⁻². s⁻¹ are applied to a culture medium comprising glucose or sodium acetate and calcium.

The culture medium can contain other elements, such as potassium, magnesium, trace elements and vitamins.

A preferred embodiment consists of using the fed-batch culture technique, which makes it possible to keep the carbon-containing substrate at non-inhibitory concentrations, while promoting an increase in biomass.

Preferably, the glucose used in the culture medium is D-glucose or dextrose, in particular dextrose obtained from biotransformation of starch, for example from maize, wheat or potato. The starch hydrolysates consist of molecules of small size, which can also be easily assimilated by the algae, providing better development of the biomass.

According to another aspect of the invention, growth of the strains depends on the presence of calcium at high concentration in the culture medium, namely above 80 mg/L of calcium, and preferably between 120 and 190 mg/L.

According to a particularly suitable embodiment, the culture medium comprises between 3 and 10 g/L of glucose and 100 and 200 g/L of calcium.

Advantageously, such a medium makes it possible to obtain a lag time of about 18 hours, and a generation time between 3 and 4 hours.

The culture method according to the invention must be carried out at an average temperature that allows good growth of the algae, preferably between 4 and 32° C.

Under these conditions, a reduction in the culture time to less than 40 hours is observed, and the lag time and generation time are very short.

The algae obtained according to the method of the invention can be used as such as a food source, in particular as animal feed, as they are potentially rich in proteins and polyunsaturated fatty acids (e.g. EPA and DHA).

According to a preferred aspect of the invention, the culture method defined above also permits production of lipids.

This method for production of lipids is characterized in that it comprises one or more of the following steps:

i) culturing unicellular algae according to the method described above, and

ii) recovering the unicellular algae cultured in step i), and

iii) extracting the lipids from the intracellular contents of the algae recovered in step ii).

The lipids can be extracted by cellular lysis, and fractionated according to the techniques known to a person skilled in the art.

The lipids thus obtained can be used in various applications, in the form of polyunsaturated fatty acids, in particular as food supplements such as substitutes for fish oils, or in the form of triglycerides, for example for the production of biofuels.

The examples given below are for the purpose of illustrating the invention, without limiting it.

### EXAMPLES

I—Screening of mixotrophic strains of Tetraselmis sp.

1—Strains:

The strains of the genus Tetraselmis (n=78) were ordered from the international culture collections CCAP (Scotland), SAG (Germany), CCMP (USA) and CSIRO (Australia).

2—Culture Media:

The fresh-water microalgae are cultured under autotrophic conditions in liquid minimum medium MM [50 mL/L of Beijerink Solution (NH₄Cl 8 g/L, CaCl₂ 1 g/L, MgSO₄ 2 g/L), 1 mL/L of phosphate buffer (K₂HPO₄ 106 g/L, KH₂PO₄ 53 g/L), 1 mL/L of a solution of trace elements (BO₃H₃ 11.4 g/L, ZnSO₄ 7H₂O 22 g/L, MnCl₂ 4H₂O 5.06 g/L, FeSO₄ 7H₂O 4.99 g/L, CoCl₂ 6H₂O 1.61 g/L, CuSO₄ 5H₂O 1.57 g/L, Mo₇O₂₄(NH₄)₆ 4H₂O 1.1 g/L, EDTA 50 g/L), 2.42 g/L of Trizma base, pH adjusted between 7.2 and 7.4 with HCl, 1.2 mg/L of vitamin B₁ and 0.01 mg/L of vitamin B₁₂ (added extemporaneously)] and in solid medium MM (+1.5% agar).

First, the marine microalgae are cultured under autotrophic conditions in reconstituted seawater or liquid medium f/2 [NaNO₃ 0.64 g/L, KCl 0.74 g/L, NaCl or Tropic marine salt 26 g/L, CaCl₂ 1 g/L, MgSO₄ 7H₂O 1.92 g/L, NaH₂PO₄ 2H₂O 50 mg/L, 1 mL/L of a solution of trace elements (Na₂EDTA 2H₂O 4.36 g/L, FeCl₃ 6H₂O 5.82 g/L, MnCl₂ 4H₂O 2.46 g/L, ZnSO₄ 7H₂O 34.5 mg/L, CoCl₂ 6H₂O 12 mg/L, CuSO₄ 5H₂O 9.8 mg/L, Na₂MoO₄ 2H₂O 2.2 mg/L), pH adjusted between 7.2 and 7.4 with HCl, 0.1 mg/L of vitamin B₁, 0.6 mg/L of vitamin B₈ and 0.6 mg/L of vitamin B₁₂ (added extemporaneously)] and in solid medium f/2 (+1.2% agar).

The cultures of microalgae under mixotrophic conditions and under heterotrophic conditions were carried out on medium MM or f/2 with the respective addition of the following carbon-containing substrates: acetate 5 mM, glucose 5 g/L, lactose 10 g/L, saccharose 10 g/L and glycerol 5 g/L.

3—High-Throughput Screening of the Growth of Strains in Liquid Medium in 24- and 96-Well Microplates:

The heterotrophic and mixotrophic character of the strains of Tetraselmis was evaluated by culturing the strains of microalgae immediately upon receiving them in medium MM (fresh-water strain) or f/2 (marine strain) in the presence of a carbon-containing substrate in 24-well (V=2 mL) or 96-well (V=1 mL) microplates, sealed with gas-permeable film. Growth under autotrophic conditions (MM or f/2) was monitored systematically to serve as reference for the cultures under mixotrophic and heterotrophic conditions.

The microplates were placed in an incubation chamber (SANYO MLR-351H) at 22° C., 60% humidity and 10 μE of light intensity for the cultures under autotrophic and mixotrophic conditions and in an incubation chamber (BINDER KB53) at 22° C., 60% humidity and in darkness (0 μE) for the cultures under heterotrophic conditions.

The incubation chamber was modified in order to supply controlled lighting in the form of flashes of light of 10 μE at a rate of 30 flashes of 30 seconds per hour.

4—High-Throughput Screening by Spectrofluorometry of the Intracellular Lipid Content of the Strains of Microalgae Cultured Respectively Under Mixotrophic and Heterotrophic Conditions:

After monitoring growth in 24- or 96-well microplates for 3 to 4 weeks, the intracellular lipid content of the mixotrophic and heterotrophic microalgae was evaluated by spectrofluorometry. The intracellular lipids were labelled specifically with a fluorochrome, Nile Red.

In response to fluorescence excitation at 488 nm, the neutral lipids stained with Nile Red emit fluorescence at 570 nm and the polar lipids at 610-620 nm.

For this purpose, the liquid cultures of strains of microalgae carried out under mixotrophic and heterotrophic conditions are transferred to 96-well PCR microplates (V=100 to 200 μl of culture) and stained with 1-2 μl of Nile Red (0.1 mg/mL). After incubation or 20 min in darkness, the PCR microplate is placed in a spectrofluorometer (Varian) and a scan of fluorescence emission from 500 to 700 nm is carried out.

The effect of the flashes of light (autotrophy and mixotrophy columns) and of carbon-containing substrates (mixotrophy and heterotrophy columns) such as glucose (Glc 5 g/L), acetate (Ac 1 g/L), saccharose (Sac 10 g/L), lactose (Lac 10 g/L) and glycerol (Gly 5 g/L) on growth of the 78 strains of the genus Tetraselmis was evaluated by screening in 96-well microplates on liquid medium MM or f/2. The growth is monitored twice weekly for 3 to 4 weeks by macroscopic observation of the cultures and microscopic observation with a binocular microscope (10× and 32× objectives).

5—Results:

Out of the 78 strains tested of the genus Tetraselmis, 22 displayed a heterotrophic character and only 7 strains proved to be strictly autotrophic. It is in fact observed that the 22 heterotrophic strains show significant growth at 0 μE on one of the substrates tested, comparable or even greater than that under autotrophic conditions. The preferred carbon-containing substrate of the strains of the genus Tetraselmis appears to be glucose with 17 strains mixotrophic out of 21. The strains are called strictly autotrophic when the addition of a carbon-containing substrate in the presence of or in the absence of light does not improve cell growth relative to culture under autotrophic conditions. The supply of light (10 μE) and the carbon-containing substrate, in the present case, enabled 71 strains of the genus Tetraselmis to reach a cellular concentration comparable to or greater than that of the control under autotrophic conditions. Culture under mixotrophic conditions therefore constitutes the best conditions for culture of the largest number of strains of the genus Tetraselmis.

The effect of light on the accumulation of intracellular lipids was evaluated in the 21 mixotrophic strains of the genus Tetraselmis capable of growing under mixotrophic conditions in continuous and discontinuous lighting, in 96-well microplates, under stationary-phase culture conditions.

In practice, a culture aliquot (200 μl) of each of the 21 heterotrophic strains of Tetraselmis is transferred to a 96-well microplate, then stained with Nile Red (1 μg/mL). The fluorescence emission signal, reflecting the accumulation of intracellular lipids (neutral lipids at 570 nm and polar lipids at 620 nm), is collected by means of a spectrofluorometer.

For the 21 strains tested, there was more intense staining of the strains cultured in the presence of flashes, relative to those cultured with continuous illumination with Nile Red.

### II—Growth of the Mixotrophic Strains of Tetraselmis sp. in Flash Mode

1—Culture of the Strains of Tetraselmis sp. in a Bioreactor:

Two strains of Tetraselmis, taken at random from the 21 mixotrophic strains selected during the screening described in part I above, were cultured in flash mode according to the invention in fermenters (fed-batch). In parallel, the same strains were cultured under autotrophic conditions with continuous illumination.

The cultures were carried out in 2-litre fermenters (BioController ADI 1030) for use with dedicated automatic equipment and with computerized supervision. The pH of the system was adjusted by adding base (solution of sodium hydroxide at 1N) and/or acid (solution of sulphuric acid at 1N). The culture temperature is fixed at 23° C. Stirring was provided by 3 stirring rotors, mounted on the shaft according to the Rushton configuration (three-blade impellers with down pumping). The stirring speed and the aeration flow rate were regulated to a minimum of 100 rpm and a maximum of 250 rpm with Q_{min.}=0.5 vvm/Q_{max.}=2 vvm respectively. The bioreactor is equipped with an external lighting system surrounding the transparent tank. The intensity of the light emitted, as well as the light cycles were programmed from a computer station. The reactors were inoculated with a preculture carried out on a stirring table (140 rpm) in a thermostatic chamber (22° C.), illuminated continuously at 100 μE. The precultures and cultures in bioreactors were carried out in medium f/2 supplemented with 10 mM of NaHCO₃. The carbon-containing substrate that was used for the culture under mixotrophic conditions in the bioreactor is sodium acetate at a concentration of 20 mM. Starting from 92 h of culture, additions of concentrated medium f/2 were made every 24 h in order to reach a final concentration of 0.5×. For the “flash” cultures under mixotrophic conditions, 5 mM of sodium acetate was added as well as the concentrated medium f/2.

2—Supply of Light in the Form of Flashes:

Light was supplied in the form of flashes in the bioreactors using LED lamps distributed around the external wall of said fermenters. A clock switched on these LED lamps for illumination times or pulses between 8 and 50 μE. The light intensity of the flash system is equal to that used in continuous mode in the control cultures under autotrophic conditions.

3—Monitoring the Biomass:

The total concentration of biomass was monitored by measuring the dry mass (filtration on GFC filter, Whatman, then drying in a stove under vacuum, 65° C. and −0.8 bar, for at least 24 h before weighing).

4—Quantification of the Intracellular Lipids:

The quantification of the total lipids was carried out by taking samples of 10⁷ cells/mL. The method of lipid extraction is that described by Bligh, E. G. and Dyer, W. J. [A rapid method of total lipid extraction and purification (1959) Can. J. Biochem. Physiol 37: 911-917].

3—Results:

The results obtained for the various cultures are presented in the graphs in FIGS. 1 and 2.

The first graph shows a large increase in biomass when the culture is carried out in flash mode (mean value of the different mixotrophic strains selected) relative to culture of the same strains carried out in autotrophic mode.

The second graph shows an accumulation of lipids in the cells cultured in flash mode up to 30% greater than that of the cells cultured in autotrophic mode.

4—Conclusion:

The above study shows that supply of light in the form of flashes (30 flashes of 30 seconds per hour of about 10 μE) to cultures of various microalgae of the genus Tetraselmis, as well as carbon-containing substrates, induces, in all those having a mixotrophic character, a significantly greater accumulation of intracellular lipids.

In terms of biomass, the yields were much higher in mixotrophic mode with light supplied in the form of flashes than under autotrophic conditions. The concentration of microalgae obtained is between 100 and 150 g/L (FIG. 1), which is much higher than the concentrations obtained with the cultures in continuous light.

Moreover, the method for culturing unicellular algae according to the invention makes it possible to reduce the culture time of said algae to less than 40 hours, with a very short lag time and generation time.

1. Method for culturing unicellular algae cultured in mixotrophic mode, characterized in that said algae are cultured in darkness with a supply of light that is discontinuous or variable over time, the intensity of which in micromoles of photons varies with an amplitude greater than or equal to 10 μmol. m⁻². s⁻¹ at a rate of several times per hour.

2. Method for culturing unicellular algae according to claim 1, characterized in that said light intensity reaches successively the values 2 μmol. m⁻². s⁻¹ and 10 μmol. m⁻². s⁻¹ at a rate of several times per hour.

3. Method according to claim 1, characterized in that the light intensity varies cyclically.

4. Method according to claim 1, characterized in that the light intensity varies between the values 0 and 20 μmol. m⁻². s⁻¹, preferably between 0 and 50 μmol. m⁻². s⁻¹.

5. Method according to claim 1, characterized in that the supply of light is discontinuous.

6. Method according to claim 5, characterized in that the light is supplied as flashes.

7. Method according to claim 6, characterized in that said flashes consist of successive phases of illumination with a duration comprised between 5 seconds and 10 minutes, preferably between 10 seconds and 2 minutes, more preferably between 20 seconds and 1 minute.

8. Method according to claim 7, characterized in that said flashes comprise between 20 and 30 flashes per hour.

9. Method according to claim 6, characterized in that the intensity of the flashes is comprised between 5 and 15 μmol. m⁻². s⁻¹.

10. Method according to claim 1, characterized in that the light intensity varies continuously over time.

11. Method according to claim 1, characterized in that the culture medium is a minimum medium supplemented with carbon-containing substrate comprising acetate, glucose, cellulose, starch, lactose, saccharose or glycerol.

12. Method according to claim 11, characterized in that the carbon-containing substrate comprises predominantly sodium acetate.

13. Method according to claim 1, characterized in that the unicellular algae are selected from the classes: Euglenophyceae, Prasinophyceae, Eustigmatophyceae, Bacillariophyceae, Prymnesiophyceae, Pinguiophyceae, Dinophyceae, Trebouxiophyceae, Bicosoecophyceae, Katablephariophyceae, Chlorophyceae, Haptophyceae, Raphidophyceae, Chysophyceae, Coscinodiscophyceae, Alveolata, Bangiophyceae, Rhodophyceae.

14. Method according to claim 1, characterized in that the unicellular algae belong to the Prasinophyceae class, preferably to the genus Tetraselmis sp.

15. Method for producing lipids, characterized in that it comprises the following steps:
i) culturing unicellular algae according to the culture method of claim 1, and
ii) recovering the unicellular algae cultured in step i), and
iii) extracting the lipids from the intracellular contents of the algae recovered in step ii).
